(19) United States
(12) Patent Application Publication
(10) Pub. No.: US 2009/0204003 A1
(43) Pub. Date: Aug. 13, 2009

(54) TRACKING SELECTION FOR MEDICAL DIAGNOSTIC ULTRASOUND IMAGING

(76) Inventor: Ismayil M. Guracar, Redwood City, CA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/027,940

(22) Filed: Feb. 7, 2008

(51) Int. Cl. A61B 8/00

(52) U.S. Cl. 600/458; 382/128

(57) ABSTRACT

Data is captured for different contrast agent perfusion imaging options. For example, two different maximum intensity projection (MIP) combinations of frames are formed during scanning, one with motion correction and one without. As another example, the component frames of data are stored to allow any combination after scanning. If one combination is less desirable, then another combination may be provided without requiring further injection or additional scanning. In another embodiment, the sufficiency of combination is detected and the combination of frames is selected automatically as a function of ultrasound data.

### BACKGROUND

The present embodiments relate to medical diagnostic ultrasound imaging of a region over time. In particular, data is combined from scans performed at different times.

Data may be combined from a plurality of scans for imaging contrast agent perfusion. Contrast echocardiography is a widely used technique in imaging the heart. An evolving application of contrast echo is myocardial contrast echocardiography (MCE) in which the contrast microbubbles provide traces for the microcirculation and for myocardial perfusion. Imaging blood perfusion in organs or tissue may be useful. In some applications, frames of data acquired over time are integrated. The resulting image may provide useful information for diagnosis, such as showing smaller vessels or perfusion channels.

Some example combinations are maximum intensity holding/processing (MIP), minimum intensity holding, and the construction of a time intensity curve (TIC). Maximum intensity processing combines the high luminance contrast portion over time. TIC charts intensity (e.g., contrast agent frame intensity) for a pixel or region of interest as a function of time. The chart shows the in-flow, out-flow, or both of contrast agents over the time associated with the component frames of data.

Motion from breathing, heart motion, other tissue motion, and transducer motion may blur a MIP image or cause errors in a TIC. Motion tracking may stabilize the individual component images prior to the MIP operation, reducing the blurring. However, breathing motion or motion from the patient may be minimal. In these cases the motion tracking may introduce a loss of spatial resolution due to minor tracking errors from slight out of plane motion and other errors. In these situations, conventional non-tracked MIP images may be preferred.

Tracked or non-tracked MIP capture may be chosen by a user prior to the capture. The user makes a selection prior to MIP imaging based on the perceived level of tissue motion in the region of interest and the level of cooperation of the patient in breath holding. If tracked MIP is chosen and there is significant motion, then the best possible final capture is available. If there is no significant motion, then there may be some degradation over the conventional MIP capture without tracking. Similarly, choosing conventional MIP without tracking may be rewarded with a sufficient image, but may not be, depending on motion during capture of the frames of data. An additional capture sequence or possibly an additional injection may be required to achieve the best image quality.

### BRIEF SUMMARY

By way of introduction, the preferred embodiments described below include methods, systems, computer readable media, and instructions for selecting combinations of frames of ultrasound data. Data is captured for different imaging options. For example, two different MIP combinations of frames are formed during scanning, one with motion correction and one without. As another example, the component frames of data are stored to allow any combination after scanning. If one combination is less desirable, then another combination may be provided without requiring further injection or additional scanning. In another embodiment, the sufficiency of combination is detected and the combination of frames is selected automatically.

In a first aspect, a method is provided for selecting combinations of frames of ultrasound data. A sequence of ultrasound frames of data representing, at least in part, a region of a patient is generated. While or after generating, an indication of whether to correct for motion between the ultrasound frames of data is received. An image is formed as a function of the sequence of ultrasound frames of data and the indication. The forming includes correcting for relative motion between the ultrasound frames of data if the indication is to correct, and the forming does not include correcting for relative motion between the ultrasound frames of data if the indication is not to correct.

In a second aspect, a computer readable storage medium has stored therein data representing instructions executable by a programmed processor for selecting combinations of frames of ultrasound data. The storage medium includes instructions for correcting for motion between frames of contrast agent data, combining the frames of ultrasound data with the motion correcting, combining the frames of ultrasound data without the motion correcting, and generating a first image as a function of the combined frames without the motion correcting, a second image as a function of the combined frames with the motion correction, or both.

In a third aspect, a system is provided for selecting combinations of frames of ultrasound data. A processor is operable to combine a plurality of frames of ultrasound data and operable to select between motion tracking of the frames and no motion tracking of the frames. The selecting is a function of the ultrasound data. A display is operable to display an image as a function of the ultrasound data of the combination.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound imaging system for selecting combinations of frames of ultrasound data; and

FIG. 2 is a flow chart diagram of a method for selecting combinations of frames of ultrasound data according to one embodiment.

### DETAILED DESCRIPTION OF THE DRAWINGS AND PRESENTLY PREFERRED EMBODIMENTS

Simultaneous motion tracked and non-motion tracked captures permit users to later choose which combination to use for diagnosis. Motion tracked capture (tMIP) and conventional capture (cMIP) combinations are assembled in separate storage memories or formed from stored component frames. The user selects the preferred combination during the capture or after the capture is completed. Alternatively or additionally, the highest quality representation of the distribution of contrast in small vessels or tissue may be determined automatically.

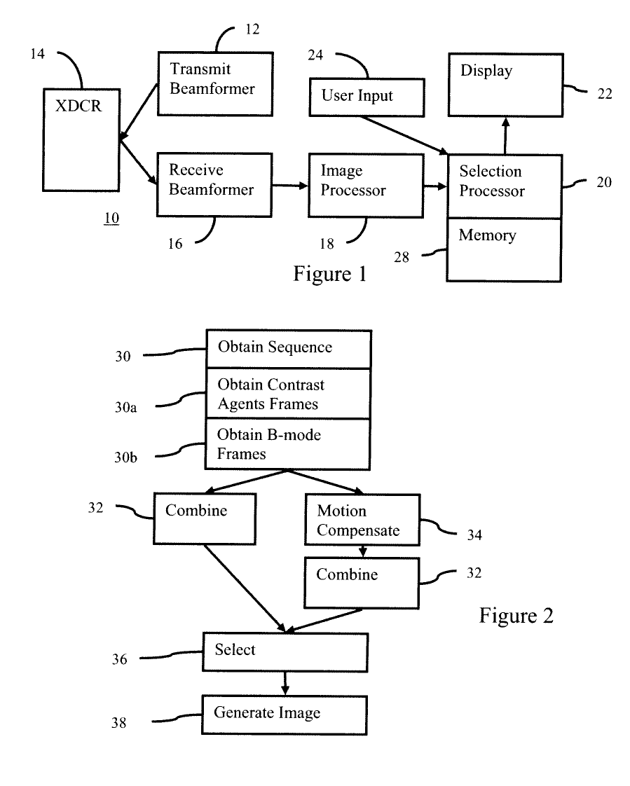

FIG. 1 shows a system 10 for selecting combinations of frames of ultrasound data. The system 10 includes a transmit beamformer 12, a transducer 14, a receive beamformer 16, an image processor 18, a selection processor 20, a display 22, a user input 24, and a memory 28. Additional, different, or fewer components may be provided. For example, the selection processor 20 is combined with or part of the image processor 18. In another example, the user input 24 is not provided.

The system 10 is a medical diagnostic ultrasound imaging system in one embodiment, but other imaging systems of the same (ultrasound) or different modality may be used. The system 10 provides real-time or offline operation. In other embodiments, part or all of the system 10 is implemented in a computer or workstation. For example, previously acquired frames of data are processed without the beamformers 12, 16 or transducer 14. Offline software on the computer or workstation implements the selection of tracking between frames of ultrasound data.

The user input 24 is a keyboard, mouse, trackball, touch pad, touch screen, buttons, sliders, combinations thereof, or other input device now known or later developed. The user input 24 allows user selection of the type of image to display, the default image to be used during capture, and/or change of the image displayed.

The transmit beamformer 12 is an ultrasound transmitter, memory, pulser, analog circuit, digital circuit, or combinations thereof. The transmit beamformer 12 is operable to generate waveforms for a plurality of channels with different or relative amplitudes, delays, and/or phasing. Upon transmission of acoustic waves from the transducer 14 in response to the generated waves, one or more beams are formed. The transmit beamformer 12 may cause the beam to have a particular phase and/or amplitude. For example, the transmit beamformer 12 transmits a sequence of pulses associated with a given scan line or to adjacent scan lines. The pulses correspond to beams with different amplitudes and/or relative phases. In alternative embodiments, a single beam is used for any given scan line and/or beams with a same amplitude and/or relative phases are used.

The transducer 14 is a 1-, 1.25-, 1.5-, 1.75- or 2-dimensional array of piezoelectric or capacitive membrane elements. The transducer 14 includes a plurality of elements for transducing between acoustic and electrical energies. The elements connect with channels of the transmit and receive beamformers 12, 16.

The receive beamformer 16 includes a plurality of channels with amplifiers, delays, and/or phase rotators, and one or more summers. Each channel connects with one or more transducer elements. The receive beamformer 16 applies relative delays, phases, and/or apodization to form one or more receive beams in response to each transmission. In alternative embodiments, the receive beamformer 16 is a processor for generating samples using Fourier or other transforms.

The receive beamformer 16 may include a filter, such as a filter for isolating information at a second harmonic or other frequency band relative to the transmit frequency band. Such information may more likely include desired tissue, contrast agent, and/or flow information. In another embodiment, the receive beamformer 16 includes a memory or buffer and a filter or adder. Two or more receive beams are combined to isolate information at a desired frequency band, such as a second harmonic, cubic fundamental, or other band.

Any desired sequence of transmit and receive operation may be used to obtain ultrasound information. For example, B-mode data may be obtained by scanning a region once. The B-mode data may be used for tissue imaging. Correlation or motion tracking may be used to derive fluid information from B-mode data. B-mode operation may provide contrast agent information. Doppler information may be obtained by transmitting sequences of beams along each scan line. A corner turning memory may be used to isolate tissue, contrast agents, and/or flow information from Doppler signals. Other now known or later developed modes may be used.

In one embodiment, the mode is a contrast agent-imaging mode. Contrast agents may be imaged with typical B-mode or Doppler techniques. Isolating information at the second, even, odd, sub, or other harmonics may more likely identify information from contrast agents. For example, a two-pulse technique is used. The pulses have a same amplitude, but different phase. By summing the response, information associated with even harmonics is identified. Filtering may alternatively be used. Alternatively or additionally, relative phasing is provided in the receive processing.

In one embodiment, the transmit sequence is controlled to generate echo signals responsive to the cubic fundamental. The beamformer 12 is operable to transmit a plurality of pulses having at least two different amplitude levels and at least two of the plurality of pulses having opposite or different phases. Transmitter power can be varied in any suitable manner, as for example by adjusting the voltage applied to individual transducer elements, or by adjusting the number of transducer elements (or transmit aperture) used to form a particular pulse.

For obtaining ultrasound data at the cubic fundamental, the receive beamformer 16 includes line memories and a summer or a filter to combine signals responsive to the transmissions. The line memories or buffers can be formed as physically separate memories, or alternately they can be formed as selected locations in a common physical device. The beamformed signals are stored in the line memories or buffers and then weighted and summed in a weighted summer. Weighting values for both amplitude and phase are used in the weighted summer. The memories and the summer can be implemented using analog or digital techniques. The weighted summer forms a composite output signal by weighting the separate beamformed receive signals. The composite output signal for a given spatial location is a sample associated with the cubic fundamental response.

Obtaining cubic fundamental information is disclosed in U.S. Pat. No. 6,494,841, the disclosure of which is incorporated herein by reference. Any of the transmit sequences and receive combinations disclosed therein may be used for obtaining cubic fundamental information. Other transmit sequences and receive combinations for obtaining cubic fundamental information may be used, such as disclosed in U.S. Pat. Nos. 6,602,195; 6,632,177; 6,638,228 and 6,682,482, the disclosures of which are incorporated herein by reference. In general, a sequence of pulses with different amplitudes and phases is transmitted. Using amplitude change or different amplitudes without different phases may also be used to obtain cubic fundamental information. By combining received signals responsive to the sequence, a sample including cubic fundamental information is obtained. The cubic fundamental information is highly specific to ultrasound contrast agents since contrast agents produce cubic response and the transducer and tissue produce very little cubic response. The information provides tissue clutter rejection, allowing for imaging more specific to contrast agents. For example, small vessels within tissue may be more easily imaged or identified using cubic fundamental information.

The image processor 18 is a B-mode detector, Doppler detector, pulsed wave Doppler detector, contrast agent detector, correlation processor, Fourier transform processor, application specific integrated circuit, general processor, control processor, field programmable gate array, digital signal processor, analog circuit, digital circuit, combinations thereof, or other now known or later developed device for detecting information for display from beamformed ultrasound samples. Any now known or later developed detector may be used. The detector outputs a plurality of frames to be combined. Any type of data may be combined, such as B-mode data, Doppler data, and/or contrast agent data.

In one embodiment of a contrast agent detector, the image processor 18 implements a fast Fourier transform from a plurality of samples representing a same region or gate location. Each of the samples is responsive to cubic fundamental so that a pulsed wave Doppler display may be generated from cubic fundamental information. Any of the contrast agent detectors in the patents referenced above may be used. Other components may be used for a contrast agent detector. For example, B-mode detection is provided. As another example, a filter combines information from different transmissions to enhance or better isolate the response from contrast agents (e.g., second harmonic or cubic fundamental). The filter obtains information primarily at a cubic fundamental or other frequency band of the transmitted ultrasound signals. Any detection of the signals is then performed.

The image processor 18 also includes a B-mode detector in a parallel track. The B-mode detector operates on the same or different beamformed samples to detect tissue, contrast agent, or tissue and contrast agent response. For example, one receive beam for each spatial location from the sequence of receive beams used for cubic fundamental isolation is applied to the B-mode detector for imaging primarily tissue information.

The image processor 18 outputs frames of ultrasound data. The frames of data are formatted in an acquisition format (e.g., polar coordinate), a display format (e.g., scan converted into a Cartesian coordinate format or an image), or other format. Each frame of data represents a one, two, or three-dimensional scanned region, such as substantially the entire region to be imaged (substantially accounting for patient or transducer motion). The frames of data include a single type or multiple types of data. For example, one frame of data includes just contrast agent information. As another example, one frame of data includes contrast agent information for some spatial locations and another type of information (e.g., B-mode or Doppler) for other spatial locations. Different types of data may be provided in the same frame for a same spatial location. In another example, the different types of data are provided in different frames of data.

In an alternative embodiment, the image processor 18 loads data from a network or memory. For example, to acquire or obtain ultrasound data, DICOM or other images are loaded. Each image is a frame of data. One frame may include different types of data, one overlaid on another. Alternatively, each frame includes only one type of data with different frames for different data types. In another embodiment, each frame is subdivided so that one portion includes one type of data and another portion includes another type of data.

The selection processor 20 is an application specific integrated circuit, correlation processor, Fourier transform processor, general processor, control processor, field programmable gate array, digital signal processor, analog circuit, digital circuit, combinations thereof, or other now known or later developed device for selection associated with combinations of data. The selection processor 20 generates different combinations of data to allow selection of the desired combination and/or selects a combination to be formed as a function of ultrasound data.

In one embodiment, the different combinations are distinguished by tracking. For tracking, the selection processor 20 spatially aligns frames of data output from the contrast agent detector. The frames of data may be spatially aligned based on external sensors, such as transducer position sensors. The frames of data may be spatially aligned based on the contrast agent data. In one embodiment, the frames of contrast agent data are spatially aligned as a function of frames of data output from the B-mode or other non-contrast agent detector. The B-mode frames are acquired at the same or similar times as the contrast agent frames, such as in a line or frame interleave. Aligning the B-mode frames indicates alignment of the contrast agent frames acquired at the same or similar times.

One combination of frames uses data from motion corrected frames and the other combination is formed without tracking. The selection processor 20 may include a persistence filter, other filter, summer, alpha blending buffer, other buffer, memory, processor, adder, or other device for generating a combination from information of different frames of data. Separate memories or memory space may be used for the different combinations. In another embodiment, the component frames of data are stored (e.g., Cine memory) for later determination of the desired combination.

The selection processor 20 combines the spatially aligned and non-spatially aligned frames of data output from the contrast agent detector in two different combinations in real time or after scanning is complete. For example, the selection processor 20 compares data for a particular spatial location from one frame to another frame or an ongoing combination frame. Based on the comparison (e.g., highest value, contribution to mean value, or lowest value), one of the values is selected or the ongoing combination frame is updated to include the desired value. As another example, the selection processor 20 determines an average, total, or other value representing a location or region as a function of time. Combinations of imaging types may be used.

The selection processor 20 selects between motion tracking of the frames and no motion tracking of the frames. Both combinations may be created, but one is selected for imaging. Alternatively, the selection processor 20 selects creation of the combination as a function of the frames of data to be used in the combination. For example, a frame associated with substantial motion is added to the combination after motion tracking, but a frame associated with little or no motion is added to the same combination without motion correct.

The selection is a function of user input. For example, the user or processor 20 selects display of one combination as the frames are acquired. During the acquisition of the sequence or after completion, the other combination may be displayed in response to user selection.

In another embodiment, the selection processor 20 selects as a function of the ultrasound data. The combinations may be compared to determine the desired combination. For example, the combination with the highest spatial frequency, lowest average intensity, or least number of pixels above a threshold is selected. As another example, the quality of motion tracking indicates the combination to be used. If the best matches are sufficiently poor, the quality of the motion correction may be poor. Accordingly, the combination without tracking may be selected. The motion amplitude may be used, such as selecting the combination with tracking where the average motion is above a threshold amount. Combinations of data processing may be used to select the combination.

The display 20 is a CRT, monitor, LCD, flat panel, projector or other display device. The display 20 receives display values for displaying an image. The display values are formatted as a one-dimensional image, two-dimensional image, or three-dimensional representation. In one embodiment, the display values are for an image generated as a function of frames of data acquired at different times, such as a TIC or MIP image. An image of the combined frames of data output from the contrast agent detector is generated. As additional frames of data are acquired and selected, the image may be updated. Other images, such as images from single or component frames of data, may also be displayed. Images from different combinations may be displayed at a same or different times.

The image processor 18 and/or selection processor 20 operate pursuant to instructions. The memory 28 is a computer readable memory. A computer readable storage medium stores data representing instructions executable by one or both of these programmed processors for selecting combinations of frames of ultrasound data. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

FIG. 2 shows a method for selecting combinations of frames of ultrasound data. The method is implemented by the system 10 of FIG. 1 or a different system. The method is performed in the order shown or a different order. Additional, different, or fewer acts may be provided, such as not providing act 30b, act 30a, and/or act 38.

In act 30, a sequence of ultrasound frames of data is obtained. The sequence is generated by acquiring frames of data with ultrasound or by acquiring previously generated frames of data (e.g., DICOM images). The frames of data are acquired in real time with live scanning or are from stored clips. The sequence may be substantially continuous or periodic (e.g., acquired once or more every heart cycle).

The sequence includes frames of data representing a scanned region at different times. Each frame of data represents a same or overlapping region. Some frames may represent different regions, such as due to out-of-plane motion of the transducer relative to the patient.

The region includes contrast agents or an area likely to include contrast agents after insertion of the agents. The contrast agents respond to ultrasound energies. Frames of contrast agent data are obtained in act 30a. Some or all of the frames of data include information from contrast agents. The information may also include response from tissue or fluids. In one embodiment, the information is obtained at a cubic fundamental of ultrasound signals. For example, ultrasound signals are transmitted in a plurality of pulses having at least two different amplitude levels and phases. To avoid or minimize destruction of the contrast agents, low amplitude transmissions (e.g., MI less than 0.7) are used. Signals responsive to the transmissions are combined, such as by summing or weighted summing. The combination provides data primarily responsive to contrast agents. Data is acquired at each spatial location of a region of interest in each frame of data.

Only one type of data is represented in the frames of data, such as data representing just contrast agents or responses from contrast agent and tissue. Alternatively, the frames of data represent different types of data, such as in a same frame or in different sets of frames. For example, frames of contrast agent data and separate frames of B-mode data are obtained. Frames of B-mode or tissue information are obtained in act 30b. The B-mode information is generated separately from the contrast agent information. Alternatively, echo signals responsive to one of the pulses (e.g., the full or highest amplitude pulse) used for contrast agent information are used for B-mode detection. The B-mode or tissue information may include other information. For example, pulse sequences and/or filtering provide for tissue information from ultrasound signals at a fundamental, second harmonic, or both.

Frames within the sequence may be discarded or not used based on characteristics. For example, the frames of data associated with less inter frame motion are selected, and frames of data associated with more inter frame motion are not selected. The frames of data with undesired motion are discarded. Any desired threshold may be used. Other criteria may be used, such as using frames from a given cycle of the heart.

In acts 32 and 34, two or more combinations of the frames are performed. The combinations are performed as the frames are acquired. Alternatively, one of the combinations is performed and the other is not, such as where automated selection is provided.

One combination is performed with motion correction in act 34. The other combination is performed without or with different motion correction. In act 34 of the combination with motion correction, frames are spatially aligned to compensate for motion.

Motion compensation of act 34 may be applied to the frames of data to correct for in-plane motion between frames. In-plane motion may be due to transducer movement, patient movement, and/or movement of the tissue within the region of interest. To compensate for motion, a relative translation and/or rotation along one or more dimensions is determined. Data from one frame is correlated with different regions in the other frame of data to identify a best or sufficient match. Correlation, cross-correlation, minimum sum of absolute differences, and/or another measure of similarity may be used. Global or local motion may be corrected. For example, the motion between the frames for a plurality of different regions is determined. A global motion may be determined from the plurality of motion corrections, or the motion correction may be applied separately for each region, warping the frame. Rigid or non-rigid motion models may be provided, such as warping in additional to translating and rotating in a non-rigid motion model. The entire frame of data or a window of data may be used for determining the best match and corresponding motion.

For each new frame of data, the previous or temporally adjacent selected frame of data is used as the reference frame. Alternatively, the same reference frame is used for comparison to each subsequent, even temporally spaced, frames of data.

The displacement of the data between frames is then used to align the spatial locations between frames. The motion correction may remove or lessen motion associated with transducer movement, patient movement, or organ movement. Alternatively, no motion correction between frames is used.

Motion sensors may be used to determine motion compensation. For ultrasound data based correction, the frames of ultrasound data may be used. In one embodiment, frames of B-mode data are used to determine the alignment of the frames of contrast agent data. The correction is performed as a function of the B-mode data. Given the contrast frames, the corresponding frames of B-mode or tissue response data are aligned by estimating the motion parameters between them using a rigid motion or non-rigid motion model. The similarity between B-mode or tissue frames indicates the alignment of the B-mode or tissue frames. Where the aligned frames are acquired at substantially a same time as the contrast agent frames, the alignment of the B-mode or tissue frames also indicates a spatial alignment of the contrast agent frames. By motion correcting using B-mode, errors in alignment due to contrast agent motion may be avoided. Since contrast agents may be moving, motion correction based on contrast agent response may be inaccurate.

In act 32, the frames of data are combined. The frames of ultrasound data are integrated as a function of time. Integration includes mathematical integration or forming an image from a plurality of sources. By combining information from contrast agents, such as information primarily at a cubic fundamental of ultrasound signals, the perfusion of contrast agents and/or small vasculature may be viewed more easily.

For each spatial location of a region of interest or all the spatial locations represented by the frames, the data is compared or used to determine a value. For each pixel of the image, a value is selected as a function of data from each of the remaining frames of data. The combination is for any now known or later developed inter-frame processing, such as maximum intensity holding, minimum intensity holding, mean determination, or constructing one or more time intensity curves.

For example, the mean, median or other statistical value of data is determined from the frames for each spatial location as a function of time. In one embodiment, the contrast agent data is combined by averaging the frames of contrast agent data. The ultrasound frames of data are averaged, such as with a weighted moving average. Consider I(Pᵢⁿ) to be the intensity of point Pᵢⁿ in the n^{th} frame. The weighted moving average of length k (k points weighted moving average) for point Pᵢⁿ is computed as follows:

$$MovingAverage ( I ( {P}_{i}^{n} ) ) = \frac{( \begin{matrix}{w}_{n} I ( {P}_{i}^{n} ) + {w}_{n - 1} I ( {P}_{i}^{n - 1} ) + … + \\ {w}_{n - k + 1} I ( {P}_{i}^{n - k + 1} )\end{matrix} )}{( {w}_{n} + {w}_{n - 1} + … + {w}_{n - k + 1} )}$$

where wₙ, wₙ₋₁, wₙ₋ₖ₊₁ are weighting coefficients. The moving window defines a number of the most recently acquired frames to include in the average, such as the most recent three frames of data. For example, a three frame moving average is provided. Infinite or finite impulse response averaging may be used. In an alternative embodiment, a weighted average of all of the frames is provided.

Temporal averaging can provide noise reduction for the image sequence in general. In contrast enhanced ultrasound image sequence, the speckle pattern at a given location may change due to the motion of the contrast agents. As a result, temporal averaging may reduce the speckle noise in this respect.

As another example combination, the maximum, minimum, or other data in relation to data of the selected frames is selected based on comparison. The frames are combined into a persisted frame or single frame. For example, a maximum intensity projection frame of data is formed from the frames of contrast agent data. A maximum of the information from contrast agents is selected for each spatial location of the image or a region of interest. Consider I(Pᵢⁿ) be the intensity of Point Pᵢⁿ in the n^{th} frame. The maximum intensity projection (along the time axis) of point i is computed as follows:

MIP(I(Pᵢⁿ))=max(I(Pᵢⁿ),I(Pᵢⁿ⁻¹),I(Pᵢⁿ⁻²), . . . ,I(Pᵢ¹))

In myocardial contrast echocardiography, contrast agents show the time trace of the myocardial perfusion. Since the contrast agents are moving over time, it is desirable to integrate all the time variant information together for better depiction of the myocardial perfusion.

In another example of combination, a curve representing intensity or other contrast agent response as a function of time is determined from the frames. The curve is for a region or for a spatial location. Since the frames are associated with different times, the curve is of intensity as a function of time.

One of the combinations is of motion corrected frames of ultrasound data. Another one of the combination is of frames of data without the motion correcting. The relative motion between the ultrasound frames of data is not corrected prior to the combination. If both captures or combinations are performed, the different combinations are available for imaging if one does not yield the desired results. This may avoid additional scanning or injections of contrast agent. User versatility is provided. Automatic selection or user selection may be provided. Maintaining both combinations or the data used to make the combinations may allow for later changes of the image used for diagnosis.

In other embodiments, additional or different simultaneous captures or storage of component frames of data for additional or different combinations is provided. Any parameter may be varied for the different combinations. For example, a maximum intensity projection and a different combination is performed. The other combination may be a persisted or averaged combination. Different averaged combinations may be provided, such as associated with different levels of recursive contribution. The combinations may or may not be performed with motion tracking.

Different combinations may be provided for differences in motion tracking. For example, one combination is associated with a more constrained motion tracking than another combination. For example, the motion tracking of one combination is constrained to just translation and not any rotation. Different size search spaces for tracking may be used, potentially permitting a greater range of transducer or patient movement. Combinations of motion constraint and search space constraint may be provided, such as no rotation but a larger search space for one combination and rotation but a smaller search space for another combination. There could be reduced processing demands with the two constrained tracking methods versus a single tracking method that is less constrained.

Any of these captures may be available for viewing at the end or be automatically selected for display based on some measure of image quality (i.e. spatial frequency content, average brightness, or pixel coverage). In other embodiments, the availability is limited to a single combination, but the combination is based on a data-based selection of the desired type of combination.

In act 36, one or more combinations are selected. The selected combination is used to generate an image. Multiple combinations may be selected for imaging at a same time or sequentially. The imaging may be performed as the frames of the sequence are acquired and/or after the frames are acquired. Alternatively, a type of combination is selected for imaging during or after acquisition of the frames of the sequence.

The selection is performed by the user. Alternatively, the selection is adaptive or as a function of ultrasound data.

For making the selection, an indication of whether to correct for motion between the ultrasound frames of data is received. The indication may be to correct for motion. Where both combinations with and without motion correction are available, the indication dictates the selection of the combination. Alternatively, the indication dictates the combination to be performed. The indication is received while generating or after generating the combination.

In one embodiment, the indication is received as user input. The user selects the combination to be used at a given time. For example, the user selects the initial combination to be displayed while scanning, switches to a different combination during the scanning, or selects a different combination after completing the scan. The user may select multiple combinations.

In another embodiment, the indication is generated as a function of data processing. Automatic selection of the combination is provided. The contrast agent data, the combined frames with motion correction, the combined frames without motion correction, the motion correction, combinations thereof, or other data is processed to determine the indication.

For example, the same frames of data are combined in two different combinations, one with motion corrected frames and the other without. The resulting combined frames of data are compared. Any comparison may be used. For example, the spatial frequency content of the combinations is compared. The spatial frequency content of the tracked and non-tracked MIP images (tMIP and cMIP) may indicate blurring. Spatial frequency is determined by high pass filtering the data, and averaging the results over a region or the entire combination frame. Alternatively, a Fourier transform is performed to identify the amplitude of the content at a desired frequency band. The combination with the higher spatial frequency content is selected. Higher spatial frequency indicates more content and less blurring. As another example, the average intensities of the combinations are compared. The average pixel intensity for both the cMIP and tMIP images is computed. The combination with the smaller average value represents less blurring or smearing due to motion. In another example, the number of pixels above a threshold or representing contrast agents is compared. The combination with the least number of pixels represents less blurring.

As another example of adaptive selection, the combination is selected as a function of a motion characteristic. The motion between the ultrasound frames of data is determined, and the indication is generated as a function of a characteristic of the motion. For example, the tracking quality is monitored. If the best match indicates a similarity worse than a threshold (i.e., the best match is a poor match), then the combination without correction is indicated. As another example, the extent of motion indicates the combination. If there is substantial motion, then the tracked capture is selected.

Combinations of indications may be used. For example, large motion may indicate using tracking, and no motion may indicate using the no tracking combination. For motion amplitudes in between, a different data characteristic may be used, such as the quality of tracking or spatial frequency.

In another embodiment, one combination is generated using different combinations. Portions of the capture (i.e. different time segments) are from different tracking types. When tracking quality allows, the non-tracked capture data is added to the combination. The combination may also include tracked segments such that the overall, final, capture image has the best available tracking type from each of the time segments. The selection is performed based on each new frame of data. If the frame of data is associated with an indication to use tracking (e.g., large motion and/or good match), then the frame is motion corrected and added to the ongoing combination frame. If the frame of data is associated with an indication not to use tracking, then the frame is added to the ongoing combination frame without motion correction. This one combination may be generated without other available combinations or is provided as one of multiple combinations for further selection.

The type of combination or capture type may be varied spatially. Different pixels, spatial locations, and/or regions of the combined frame of data may be formed differently. The indication or combination selection is performed independently for the different spatial locations. For example, the measure of tracking or registration quality varies spatially. One region may show sufficient quality to allow motion correction and another not. For the one region, motion correction is performed for the combination, but no motion correction is performed for the other region. This might be useful for cases of probe rotation where the tracking quality varies greatly throughout the image.

In act 38, an image is formed from the selected combination or combinations. The image is formed as a function of the ultrasound frames of data and the indication. The indication controls how to combine frames and/or which combination to use for imaging. For example, a combination is selected based on the indication. The image is a grey scale or color image formed from the combined frames without the motion correcting, the combined frames with the motion correction, or both.

The image is generated as a function of the combination. The frame of data formed by combining data from other frames is scan converted, color coded, mapped, and/or converted into a display format. For example, the contrast agent data is color mapped. The combination may be combined with other ultrasound information, such as being overlaid on a tissue image. Red, blue, green (RGB) or other display values are formed from the contrast agent data. Other images, such as B-mode images, may be generated. The images are grayscale, color, or combinations thereof.

The method may be repeated. Automatic selection to form one combination may be repeated with or without a repetition of injection of contrast agents. The selection and generation of the image in acts 36 and 38 may be repeated without acquiring frames of data by scanning. Since different combinations are available, different images may be generated without requiring further injection or scanning.

The images represent a two-dimensional region of the patient. In another embodiment, the images are rendered from data representing a volume. 3D imaging with motion tracking in 3D is provided. The integration or MIP operation extends to a volume. Simultaneous acquisition of tracked and non-tracked volumes may provide similar benefits to improve image quality for two and three-dimensional imaging. Since the search space in 3D is larger, there may be greater benefits to multiple constrained tracking methods being applied with the final image selected based on some measure of image quality.

Motion tracking may be improved by combining only frames of data associated with a same phase of a heart or breathing cycle. Other frame groupings may be used. Alternatively, frames from throughout one or more cycles are combined.

While the invention has been described above by reference to various embodiments, it should be understood that many changes and modifications can be made without departing from the scope of the invention. For example, the embodiments above combine contrast agent information, but other information may be combined, such as combining tissue information, without the use of contrast agents. It is therefore intended that the foregoing detailed description be regarded as illustrative rather than limiting, and that it be understood that it is the following claims, including all equivalents, that are intended to define the spirit and scope of this invention.

I (We) claim:

1. A method for selecting combinations of frames of ultrasound data, the method comprising:
generating a sequence of ultrasound frames of data representing, at least in part, a region of a patient;
receiving, while generating or after generating, an indication of whether to correct for motion between the ultrasound frames of data; and
forming an image as a function of the sequence of the ultrasound frames of data and the indication;
wherein the forming includes correcting for relative motion between the ultrasound frames of data if the indication is to correct and wherein the forming does not include correcting for relative motion between the ultrasound frames of data if the indication is not to correct.

2. The method of claim 1 wherein generating comprises obtaining the data as information representing a response of contrast agents at a cubic fundamental of ultrasound signals.

3. The method of claim 2 wherein obtaining comprises, for each ultrasound frame of data, transmitting the ultrasound signals in a plurality of pulses having at least two different amplitude levels and phases, and combining signals responsive to the transmitting.

4. The method of claim 1 wherein the frames of data represent contrast agents and the indication is to correct;
further comprising:
generating B-mode information;
wherein correcting comprises correcting as a function of similarity between the B-mode information corresponding to the ultrasound frames of data; and
wherein forming comprises combining the data representing contrast agents.

5. The method of claim 1 wherein the indication is to correct, the correcting comprising:
determining a motion displacement between the ultrasound frames of data; and
spatially aligning the ultrasound frames of data as a function of the motion displacement.

6. The method of claim 1 wherein receiving the indication comprises receiving a user input selection of the indication.

7. The method of claim 1 wherein receiving the indication comprises generating the indication as a function of data processing.

8. The method of claim 7 wherein generating the indication comprises:
combining the ultrasound frames of data with motion correction;
combining the ultrasound frames of data without motion correction; and
comparing a spatial frequency content of the combinations.

9. The method of claim 7 wherein generating the indication comprises:
combining the ultrasound frames of data with motion correction;
combining the ultrasound frames of data without motion correction; and
comparing average intensities of the combinations.

10. The method of claim 7 wherein generating the indication comprises:
determining a tracking characteristic between the ultrasound frames of data; and
generating the indication as a function of the tracking characteristic.

11. The method of claim 7 wherein generating the indication comprises:
combining the ultrasound frames of data with motion correction;
combining the ultrasound frames of data without motion correction; and
comparing a number of pixels representing contrast agents for the combinations.

12. The method of claim 1 wherein forming comprises selecting, from the ultrasound frames of data, a maximum of the information from contrast agents for each spatial location of the image.

13. In a computer readable storage medium having stored therein data representing instructions executable by a programmed processor for selecting combinations of frames of ultrasound data, the storage medium comprising instructions for:
correcting for motion between frames of contrast agent data;
combining the frames of ultrasound data with the motion correcting;
combining the frames of ultrasound data without the motion correcting; and
generating a first image as a function of the combined frames without the motion correcting, a second image as a function of the combined frames with the motion correcting, or both.

14. The computer readable storage medium of claim 13 wherein the frames of contrast agent data represent response primarily at a cubic fundamental of ultrasound signals, and wherein correcting comprises correcting as a function of frames of B-mode data.

15. The computer readable storage medium of claim 13 wherein each combining comprises forming a maximum intensity projection frame of data from the frames of contrast agent data.

16. The computer readable storage medium of claim 13 further comprising instructions for:
selecting the generation of the first image, the second image, or both as a function of the contrast agent data, the combined frames with motion correcting, the combined frames without motion correcting, the motion correcting, or combinations thereof.

17. The computer readable storage medium of claim 13 further comprising instructions for:
selecting the generation of the first image, the second image, or both as a function of user input.

18. A system for selecting combinations of frames of ultrasound data, the system comprising:
a processor operable to combine a plurality of frames of ultrasound data and operable to select between motion tracking of the frames and no motion tracking of the frames, the selecting being a function of the ultrasound data; and
a display operable to display an image as a function of the ultrasound data of the combination.

19. The system of claim 18 further comprising a contrast agent detector and a B-mode detector, the plurality of frames of ultrasound data comprising ultrasound data output from the contrast agent detector, wherein the processor is operable to motion track for the plurality of frames as a function of B-mode data output by the B-mode detector.

20. The system of claim 18 wherein the processor is operable to combine the frames of ultrasound data both as a first maximum intensity projection with motion tracking and a second maximum intensity projection with no motion tracking.

21. The system of claim 18 wherein the processor is operable to select as a function of a spatial frequency.

22. The system of claim 18 wherein the processor is operable to select as a function of an average intensity.

23. The system of claim 18 wherein the processor is operable to select as a function of a motion characteristic.
